(19) United States
(12) Patent Application Publication
(10) Pub. No.: US 2022/0143256 A1
(43) Pub. Date: May 12, 2022

(54) COMPRESSED AIR DISINFECTION SYSTEM AND METHOD OF USING SAME

(71) Applicant: Halosil International, Inc., New Castle, DE (US)

(72) Inventor: Dale KRAMER, New Castle, DE (US)

(21) Appl. No.: 17/523,183

(22) Filed: Nov. 10, 2021

Related U.S. Application Data
(60) Provisional application No. 63/112,121, filed on Nov. 10, 2020.

(51) Int. Cl. A61L 9/14

(52) U.S. Cl. CPC A61L 9/14; A61L 2209/134; B05B 12/087; A61L 2209/15; A61L 2209/111

(57) ABSTRACT

Provided herein is a system for disinfection and deactivation of contaminants, and more particularly to a self-contained compressed air disinfection system and method to deactivate pathogens on all surfaces and/or in the air of the targeted interior space comprising reusable compressed air cylinders and single-or multi dose containers of disinfectant or decontaminant.

### CROSS-REFERENCE TO RELATED APPLICATIONS

This application claims benefit of U.S. application Ser. No. 63/112,121, filed Nov. 10, 2020, which is incorporated herein by reference in its entirety.

### FIELD OF THE INVENTION

This invention pertains to disinfection and deactivation of contaminants, and more particularly to a self-contained compressed air disinfection system and method to deactivate pathogens on all surfaces and/or in the air of the targeted interior space comprising reusable compressed air cylinders and single-or multi dose containers of disinfectant or decontaminant.

### BACKGROUND OF THE INVENTION

Conventional disinfection and contaminant deactivation methods and apparatuses used therefor are generally known. The most basic of these processes including spray and wipe, along with the necessary supply of rags and disinfectants. Disinfection within interior spaces may be required for a number of reasons and is particularly important in healthcare facilities and agricultural environments to ensure infectious pathogens are not transmitted between patients or animals. For this reason, permanently, or semi-permanent installed disinfection systems may be used in these environments. It is also known to use temporary or mobile deactivation or disinfection systems in spaces that do not have permanent deactivation systems, such as gyms, hotel rooms and offices and the like.

Decontamination and disinfection of mobile spaces, such as transportation systems and mobile medical systems have been challenging. Transportation systems accommodate large volumes of travelers and commuters from a wide geographical area each day, making them critical environments in need of routine disinfection. Exemplary transportation systems in need of such decontamination and disinfection include rental and share car fleets, train cars, public transport bus, school bus, ambulance, truck, automobiles, buses, autonomous vehicles, and airplanes. However, their daily use and virtually around-the-clock operation makes disinfecting trains, buses, and airplanes challenging. As the public's concern over the spread of infection through mass transit rises, disinfection practices of transportation systems becomes ever more important to better eliminate pathogens and to safeguard the well-being of transit workers and passengers alike.

Existing mobile systems generally include a forced air supply such as a compressor, fan or turbine with: a reservoir containing deactivating or disinfecting agents, nozzles, additional fans, fluid hoses, sensors, control systems and the like, situated in a housing affixed to a mobile cart. These systems typically require an electric power source of some type. Human operators are required to operate the system.

U.S. Pat. No. 9,717,810 discloses a mobile system for treating an enclosed area with an atomized fluid comprising a compressor.

U.S. Pat. No. 7,354,551 discloses a method to microbially and/or chemically decontaminate a room such as a hotel room including a vapor generator supplying decontaminant vapor, and aeration to a level at which it is safe for occupants to enter.

In addition, it is known that deactivating and disinfecting agents can be hazardous to humans. Manual disinfection methods like spray & wipe, electrostatic or back sprayers require human operators to wear substantial personal protective clothing , and the possibility of inadequate distribution of deactivating agent disinfectant throughout the space exist by nature of human application. Further, employing whole room disinfecting systems of any kind may require human operators to delay re-entry in to a space until after the disinfectant concentration has reached 1 ppm or less, either by natural decomposition, catalytic destruction or active venting, making such processes inefficient. With the advent of the Coronavirus pandemic, the need for more efficient disinfections system has increased.

Problems exist with conventional disinfection system and method such that these systems generally require a compressor, fan or turbine, permanently installed or temporally connected to the system, an electric power source, significant manpower to operate, and are costly to install. Additional problems with these systems are their confinement to immobile spaces (such as interior rooms) and their components are suited for indoor application only. There remains a need for a system for thoroughly disinfecting rooms or transportation systems for example train cars, buses, and airplanes that enables consistent, thorough disinfection, that can be used easily without potential for alteration, and that preferably allows for single or multi dose application, fully utilizing all disinfectant, in as efficient an apparatus as possible, and reducing human applications errors. Thus, a need exists for an improved disinfection system and method, operable without a compressor, fan or turbine or electric power source, at reduced operating and maintenance cost, while operable in fixed and mobile spaces, as well as suitable for outdoor operation.

### BRIEF SUMMARY OF THE INVENTION

In accordance with an aspect of the present invention, there is provided a compressed air disinfection system for deactivating pathogens on all surfaces and/or in the air within a defined space comprising a source of a liquid disinfectant or deactivating agent, connected to a disinfectant supply line, a compressed air reservoir, connected to a high pressure air supply line, and a support member comprising, an atomizing nozzle, a first air pressure regulator, connected to an air valve, and further connected to the high pressure air supply line and an intermediate pressure air supply line to, optionally, a second pressure regulator connected to a low pressure air supply line which supplies air to the atomizing-nozzle which is also connected to the disinfectant supply line from a liquid disinfectant source, wherein the air valve is operable by a means to initiate a disinfection of decontamination cycle, and whereby the low pressure air creates a suction force to draw the liquid disinfectant or deactivating agent through the disinfectant supply line from the source of liquid disinfectant or deactivating agent to the atomizing nozzle where the airflow aerosolizes and projects the liquid into the defined space.

This system may be a multiple or single dose system in which the nozzle and system are configured so as to disperse the entire contents of the liquid disinfectant or deactivating agent. The system may also contain a control system detecting operating conditions inside the space connected to a data logging device powered by a rechargeable battery. The support member houses the nozzle assembly, pressure regulators, control system and data logging device, various sensors and connectors.

In accordance with another aspect of the present invention, there is provided a compressed air disinfection system for deactivating pathogens on all surfaces and/or in the air within a defined space comprising a single source of air and a liquid disinfectant or deactivating agent, connected to an a hydraulic nozzle.

In accordance with another aspect of the present invention, there is provided a method of deactivating contaminants within a defined space comprising installing one or more compressed air disinfection system and initiating a disinfection or decontamination cycle.

Depending on the size of the space to be disinfected, a multitude of systems may be employed. The defined space to be treated, disinfected or decontaminated may range from 1 m³ to 10000 m³, including every size within this range (e.g., from 5 m³ to 300 m³, or 300 m³ to 3000 m³ and so on). The system includes a set of sensors to detect operating conditions inside the defined space connected to a control system detecting operating conditions inside the space comprising a data logging device. The data logging device is powered by a rechargeable battery and equipped to track, among other things, identification of the space being treated, duration of treatment, disinfectant concentration in the air, nozzle pressures and temperature and humidity during the treatment cycle. The control system comprises the activation and deactivation of the data logging process, a warning system such as a flashing light, sound device or the like to alert operators about space currently being treated. The method comprising providing a compressed air disinfection system, comprising a single or multi dose of disinfectant to treat the defined space and compressed air; adjust the amount of compressed air released, so that the disinfectant is dispensed through a syphon nozzle to meet the requirements of the disinfectant for amount and speed of aerosolization to maximize efficacy and disinfect defined space.

The liquid disinfectant or deactivating agent may include hydrogen peroxide, peracetic acid, silver, chlorine dioxide, hypochlorous acid, quaternary ammonium, alcohol and mixtures thereof, and commercially available, propriety solutions such as HALOMIST®, BINARY IONIZATION TECHNOLOGY (BIT) SOLUTION, CUROXIDE, MB10, CHLOROX 360 SOLUTIONS, as well as other EPA registered and internationally known disinfectants.

During operation, the disinfectant concentration in the air may be from at least from about 1 ml/m³ to less than about 20 ml/m³ (e.g., at least 2 ml/m³, at least 3 ml/m³, at least 4 ml/m³, at least 5 ml/m³, at least 6 ml/m³, at least 7 ml/m³, at least 8 ml/m³, at least 9 ml/m³, at least 10 ml/m³) and less than about 20 ml/m³ (e.g., less than 19 ml/m³, less than 18 ml/m³, less than 17 ml/m³, less than 16 ml/m³, less than 15 ml/m³, less than 14 ml/m³, less than 13 ml/m³, less than 12 ml/m³, less than 11 ml/m³). Preferably the disinfectant concentration in the air may be from 5 ml/m³ to 15 ml/m³, more preferably from 7 ml/m³ to 12 ml/m³, and most preferably from 10 ml/m³ to 12 ml/m³. The droplet size distribution of an aerosol formed from the disinfectant solution may be from 2 to 20 nm, or from 3 to no more than 10 nm or about 5 nm with a narrow droplet size distribution. Generally, these droplet size distributions will result in droplets with large surfaces that can float in the air and are capable of evaporating rapidly. Providing and dispensing a single or multi, pre-determined dose of disinfectant to treat the defined space based on room volume and target concentration thus eliminates operator error while ensuring sufficient decontamination and disinfection.

During operation, the temperature of the space to be treated may be greater than 0° C., (e.g., greater than 5° C., greater than 10° C., greater than 15° C., greater than 20° C.) unless the disinfectant is formulated for lower temperatures and less than 100° C. Further the relative humidity before the start of the treatment should be between 20-60%.

It is an advantage that the instant system is extremely quiet when compared to systems using compressors, fans or turbines. During operation, the noise emitted from the system is about from about 40 db to about 70 db, preferably from 40 db to about 60 db as determined by sound level meter.

In accordance with another aspect of the present invention, there is provided a method of distributing one or more self-contained compressed air disinfection systems to a multitude of adjacent spaces. The method comprises providing a motorized or human powered distribution cart, and loading the cart with one or more self-contained compressed air disinfection system.

An advantage of the present invention is that it provides a disinfection system that is highly mobile and self-contained, usable where compressors, fans or turbines cannot operate due to a lack of electrical or combustion, such as generator power, or a lack of space, which reduces operator error and increases efficiency.

The system may be partially or completely operated from outside of the vehicle thus preventing any operator exposure of disinfectants.

The system may be locked in place during operation to deter unauthorized manipulation and theft.

The present invention combines the disinfectant dispersant system with a transportation conveyance that allows for a single operator to treat multiple vehicles in rapid succession. The treatment itself is completely automatic and does not require any human intervention.

The invention provides such a disinfection system and method. These and other advantages of the invention will be apparent from the description of the invention provided herein.

### BRIEF DESCRIPTION OF THE DRAWINGS

FIG. 1 shows an exemplary compressed air disinfection system useable, for example, for a through-wall application.

FIG. 2 details components housed in the support member for example, for a through-wall application.

FIG. 3 shows a compressed air disinfection system distribution cart.

FIG. 4 shows a compressed air disinfection system installed within wall in an exemplary through wall application.

FIG. 5 shows a compressed air disinfection system resting on a stand.

FIG. 6 shows a compressed air disinfection system distribution cart, including stands.

### DETAILED DESCRIPTION OF THE INVENTION

As used herein, the terms deactivation, disinfection, decontamination and sterilization are used as would be understood to the ordinary skilled artisan synonymously. The term pathogens as used herein includes, but is not limited to, biological and chemical contaminants, including, e.g., viruses, bacteria and spores.

Referring now to the drawings wherein the showings are for the purpose of illustrating a preferred embodiment of the invention only, and not for the purpose of limiting same.

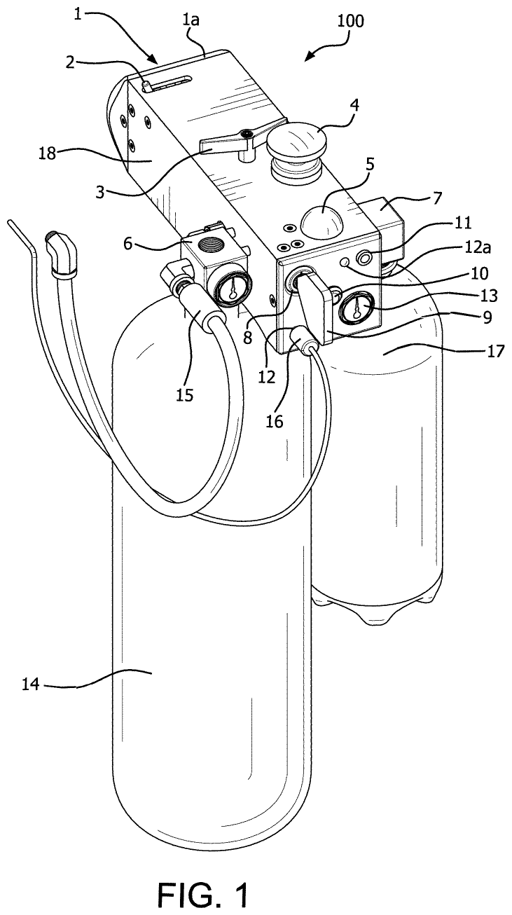

FIG. 1 shows a compressed air disinfection system 100, illustrating an embodiment of the present invention. The system comprises a source of a liquid disinfectant or deactivating agent (17), such as a bottle, canister, container, cylinder, tank or like, securely fastened into a disinfectant source socket (7); and a compressed air reservoir (14), such as a compressed air tank, cylinder, bottle or container, securely fitted into an air cylinder poppet/check valve with high pressure burst disk and pressure gauge (6). A port (15) allows for connection to a high pressure line to refill the compressed air reservoir. The disinfectant source socket (7); and the air cylinder poppet/check valve with high pressure burst disk and pressure gauge (6) are securely fastened to support member (18). The support member (18) may be of various known geometrical shapes, including generally rectangular, square, round and the like.

The support member (18) may be constructed for wall-through applications, where the wall separates the space to be disinfected or decontaminated from areas not to be treated. The portion of the support member (18) located within the designed space to be treated houses an atomizing nozzle (1) protected by a tamper proof cap (1a). The atomizing nozzle maybe a syphon nozzle, a gravity-fed nozzle, an internal or external mix, nozzle, or a hydraulic nozzle.

The source of a liquid disinfectant or deactivating agent optionally contains a sufficient amount of disinfectant to disinfect the targeted room or space at least once. Preferably, the source of the liquid disinfectant or deactivating agent, e.g., the bottle, canister, container, cylinder, tank or like, is so dimensioned that the amount of disinfectant contained therein is sufficient for exactly a single disinfection or decontamination treatment of the targeted room or space. The compressed air reservoir is so dimensioned that the amount of disinfectant is consumed before the compressed air reservoir is emptied. Optionally, the source of the liquid disinfectant or deactivating agent is configured to be single use or reusable and refillable.

A locking mechanism (2) secures the support member (18) at treatment location or during transport to a designated location, and a safety latch (3) ensures operations only when attached properly to the treatment location. For manual operation, the system may be equipped with a means (4) to initiate the start of the disinfection process or treatment cycle. However, the system's operation may be initiated remotely. The support member (18) further houses controls and indicators enabling safe operation of the system. These include an omnidirectional warning light (5). The omnidirectional warning light (5) may remain active during treatment in addition to any time determined to be useful for decomposition of disinfecting agent. The warning light may have a minimum of 180 degree field of view. For example, the omnidirectional warning light (5) may be set to remain active for a period from 5 minutes to 5 hours for hydrogen peroxide decomposition. Other operating indicators housed in the support member (18) may include a keyed locking mechanism activator (8) along with a key (9) for engaging locking mechanism, a low temperature warning light (10), a pressure gauge indicating the nozzle air pressure (13), and a high humidity indicator (11).

The support member (18) further may have a receptacle (12) for receiving a connector to charge a data logger battery, and may be further equipped with a lighted charging indicator (12a) as well as other indicators and controls useful in the operation of the system, such as a control system detecting operating conditions inside the space connected to a data logging device, a data logging device powered by a rechargeable battery.

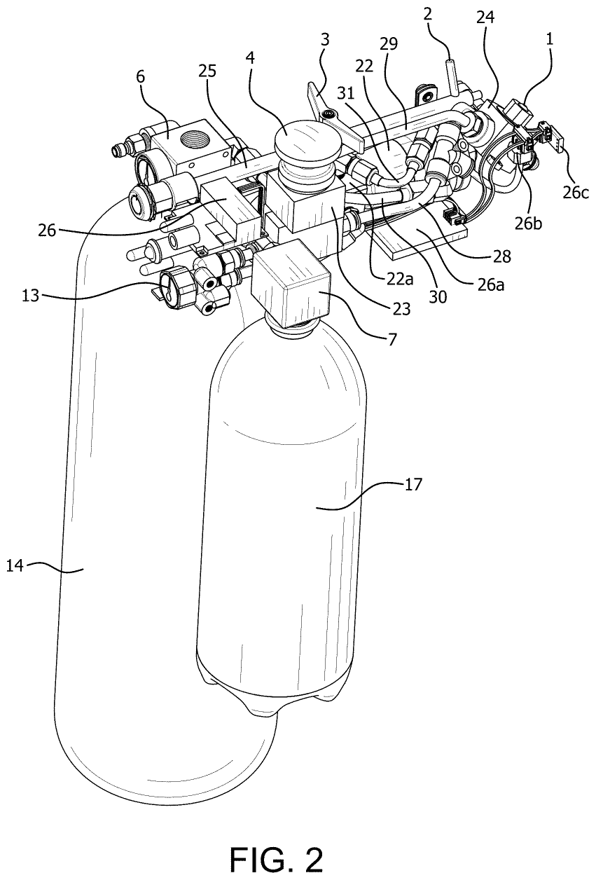

FIG. 2 shows components housed within the support member (18) for a through-wall application. An atomizing nozzle (1) housed in a nozzle body (24) is connected via a supply line providing low pressurized air (28) and a supply line providing disinfectant (29). The low pressurized air supply line (28) is in turn connected to an air valve (23) which may be actuated by a means (4) to initiate a disinfection of decontamination cycle. Such means include mechanical means such as buttons, and on/off or toggle switches. The air valve (23) may also be actuated manually or remotely or by any other means known to a person of skill in the art. High pressure air from the compressed air reservoir (14) is connected by high pressure air supply line (30) to a first air pressure regulator (22). The first air pressure regulator may be a balanced high air pressure regulator. An intermediate air pressure supply line (31) may connect the output of the first high air pressure regulator (22) to, optionally, a second pressure regulator (22a). The output of the second regulator (22a) is connected to the nozzle air pressure gage (13) which is in turn connected to the air valve (23) which is in turn connected to the atomizing nozzle via low pressure air supply line (28) which provides the pressurized air at nozzle pressure.

High pressure air may be pressurized from at least from 500 psi to 10000 psi, (e.g., at least from 1000 to 9000, at least from 1500 to 9000, at least from 2000 to 9000, at least from 3500 to 9000, at least from 3000 to 9000, at least from 3500 to 8000, at least from 4000 to 7000, at least from 4500 to 6000), preferred 4500 psi) and intermediate pressurized air may be pressurized form at least from 100 to 500 (e.g., at least from 110 to 400, at least from 120 to 300, at least from 130 to 200). The target pressure in the high pressure vessel may be dependent on the high pressure vessel volume and desired operating time. Preferably, the high pressure air is pressurized to about 4500 psi, and the intermediate pressurized air is pressurized to about 140 psi. The air pressure provided to the nozzle via line (28) maybe at least from 25 psi to 45 psi (e.g., at least from 30 psi to 40 psi, or at least from 35 psi to 40 psi). The high air pressure balanced regulator (22) is in direct communication with the nozzle air pressure regulator (22a). The locking mechanism (2, 25) may secure the support member (18) at a treatment location or during transport to a designated location. The support member (18) also may contain the data recording device logger and/or a monitor (26), an RFID reader (26a), a nozzle pressure sensor (26b), and temperature and humidity sensors (26c).

During operating of the system (100), pressurized air and the disinfecting agent, are separately routed to the atomizing nozzle (1) via pressurized air supply line (28) and a line providing disinfectant (29). Without wishing to be bound to any particular theory, if, for example a siphon nozzle is used, the energy of the air pressure intimately mixes the air and disinfecting agent within the nozzle. This creates a uniform aerosol of disinfecting agent as the mixture is sucked through the orifice and expelled as an aerosol. The pressurized air flowing through the nozzle creates a vacuum (venturi effect) and sucks the disinfectant liquid from the source of the liquid disinfectant or deactivating agent (17), to the nozzle (1).

Alternatively, a hydraulic nozzle may be used, in which the disinfectant is forced through the nozzle by high pressure, atomizing the liquid without air mixed in at the nozzle. Optionally, the source of liquid disinfectant or deactivating agent and compressed air are together, in a single reservoir, such as a cylinder, container, can, bottle, or tank. It is understood that to push the disinfectant through the nozzle, having a very small diameter orifice of about 100 microns at a rate of 50 ml per minute significant force is needed. Combining the source of liquid and air provides this force.

To further illustrate using a hydraulic nozzle, a 2.5 liter air tank may include 1.25 liters of liquid disinfectant which may then be pressurized to about 4500 psi. Turning tank air tank ‘upside down’ meaning the tank's opening is on the bottom, the disinfectant liquid will be the first flowing in the supply line connected to the nozzle at the bottom of the tank. A person of skill in the art would also understand that turning it upside down is not required if there is a pickup tube from the tank neck to the other end of the tank or even a flexible pick up tube that ‘by gravity’ always remains at the bottom of the tank where the liquid is. During operation, in this illustration, the 1.5 liters of compressed air will start pushing on the liquid at 4500 psi but by the time that the liquid is fully dispensed the air will be only pushing at 2250 psi, twice the volume and hence ½ the pressure if at the same temperature.

Optionally, when a hydraulic nozzle is used, a pressure regulator is used to maintain a constant liquid pressure at the nozzle during the entire time liquid is dispensed through the nozzle. Preferably, no pressure regulator is used.

Regulating high pressure down to a steady low pressure requires sophisticated balancing hardware, generally known to a person of skill in the art. It is understood that pressures within the system may fluctuate, but that for efficient and proper operation of the system, the pressures will need to be regulated. The balancing hardware is required to maintain a constant intermediate pressure of preferably about 140 psi during the entirety of the disinfection or treatment cycle, independently of the compressed air pressure provided. The compressed air pressure maybe as low as about 0 psi to as high as about 10000 psi, and any pressure within that range.

During operation, optimizing air and fluid pressures to ensure complete emptying of the liquid disinfectant or deactivating agent source might be advantageous. For example, if the atomizing nozzle is a siphon nozzle, to empty a source of a liquid disinfectant or deactivating agent (17) completely, the siphon must be sufficiently strong during the entire decontamination or disinfectant treatment cycle, requiring about 15-40 psi of air pressure at the nozzle. Too much or too little air pressure will impact droplet size distribution and liquid nozzle pressure. The liquid pressure at the nozzle best aerosolizes at negative fluid pressure. The liquid pressure may be from at least −0.5 psi to 0 psi, (e.g., at least from −0.4 to 0, −0.3 to 0, −0.2 to 0, −0.1 to 0, or −0.5 to 0). Maintaining a steady air pressure at the end of the cycle is critical to empty the disinfectant source and is achieved by adequate amount of compressed air and balanced air regulation. It is further understood that the complete emptying of the liquid disinfectant or deactivating agent source is dependent, for example, on the volume of liquid, the air tank internal volume, the air starting temperature, the ambient surrounding temperature, the starting pressure, the pressure drops in hoses, valves and the nozzle, the height of the nozzle venturi above the liquid level (which changes as the liquid is dispensed).

The data recording device (26), such as a commonly known data logger, may record information such as time and date of decontamination or disinfection treatment, ambient temperature, humidity, and nozzle, various operating pressures and the like. There may further be included an RFID reader (26a) that records the treatment location by reading RFID tags placed in treatment locations. It is generally known that information recorded at a location or provided by an operator may be communicated to other systems. For example, information recorded by the system may be accessible by an operator, either at the location or remotely, from a localized or central database, or via a mobile application, accessible, e.g., on a specifically designed platform or operable from a generally known mobile device.

The atomizing nozzle may be positioned within the space to be decontaminated such that an optimal trajectory of the aerosol is achieved for evaporation. For example, while positioning of the nozzle at the ceiling of the space is possible, excessive condensation may impair or delay proper functioning. Thus, positioning of the nozzle within the wall near the floor or within the floor is preferred.

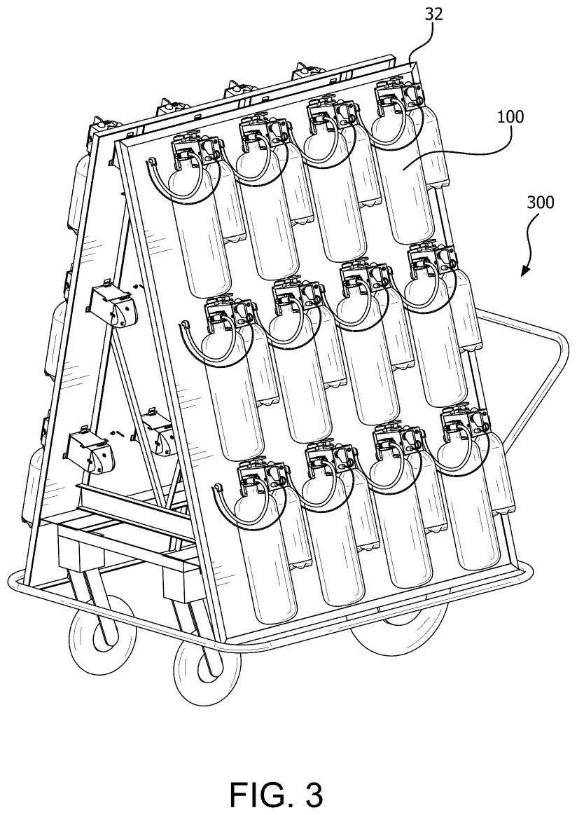

FIG. 3. shows motorized cart (300) having a panel (32) designed to receive one or more compressed air disinfection systems (100). Preferably, the panel is designed to receive a multitude of such system. The cart may be used for example during re-charging and transportation to a desired location. During transport, the compressed air disinfection system (100) is secured engaging the locking mechanism (2, 25, 8, 9)

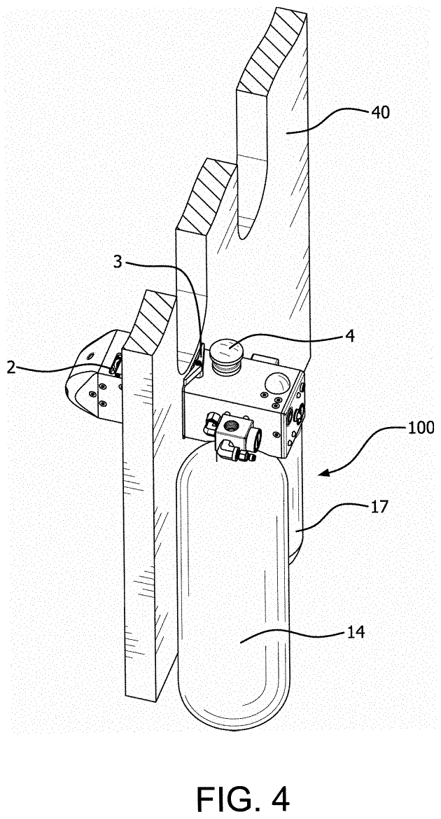

FIG. 4 shows the compressed air disinfection system (100) installed in a wall-though application. The support member (18) is inserted through the wall (40) until the locking mechanism (2) secures the system in place and the safety latch (3) is disengaged. The opening in the wall may be a door, slide, vent, or similar opening. The source of a liquid disinfectant or deactivating agent (17), the compressed air reservoir (14) and the on/off switch (4) are located outside the wall (40). The support member (18) may be installed temporarily or permanently within the wall (40). This installation, for example, allows an operator to only attach the liquid disinfectant or deactivating agent (17) and the compressed air reservoir (14) to their respective ports (6, 7), and the operator may initiate the disinfecting task from the outside, via the on/off switch (4) or remotely. The cartridges may be attached on site, after the support member placed and secured through the wall, or may be installed prior to installing the support member through the wall. Alternatively, the compressed air disinfection system may be placed inside of a room or enclosure, such as a motor vehicle, trailer or the like In that case, the shape of the support member (18) may be adapted to, for example be securely attached to a window or other internal structure of the targeted space. Suitably sized amounts of liquid disinfectant or deactivating agent (17) and the compressed air (14) may be provided for each disinfectant or treatment cycle. While amounts of deactivating agent and compressed air may be sufficient for several disinfectant cycles, amounts of deactivating agent and compressed air sufficient for disinfecting or treating a designated space exactly once are preferred. Preferably, the deactivating agent (17) and the compressed air (14) are provided in refillable bottles, containers or cylinders, and each of these containers contain at the start of each treatment cycle only enough air and disinfectant for one disinfectant or treatment cycle.

Single or multi dose cartridges for deactivating agent and compressed air for the disinfection of a designated space have the advantage that specifically measured amounts of each are provided, efficiency of operation is maximized, and operator error minimized, ensuring adequate disinfection. Alternatively ports (7, 6) allow for connection to a high pressure line to refill the compressed air reservoir, and the disinfectant or deactivating agent while the cartridges are attached to their ports and the compressed air disinfection system (100) is installed in the wall. Other advantages include extremely easy installation without the need for electrical connections, or moving parts.

The compressed air disinfection system (100) may thus be operated as partially installed system or as a completely self-contained mobile system. A partially installed system may include semi-permanently or permanently installed though-wall systems. Self-contained mobile system include for example self-propelled mobile autonomous vehicles or devices that may be employed to disinfect designated spaces, such as hotels, hall-ways, shopping malls, and like, without the need for human operators. Further, the system maybe combined with existing self-propelled systems, such as floor cleaning systems.

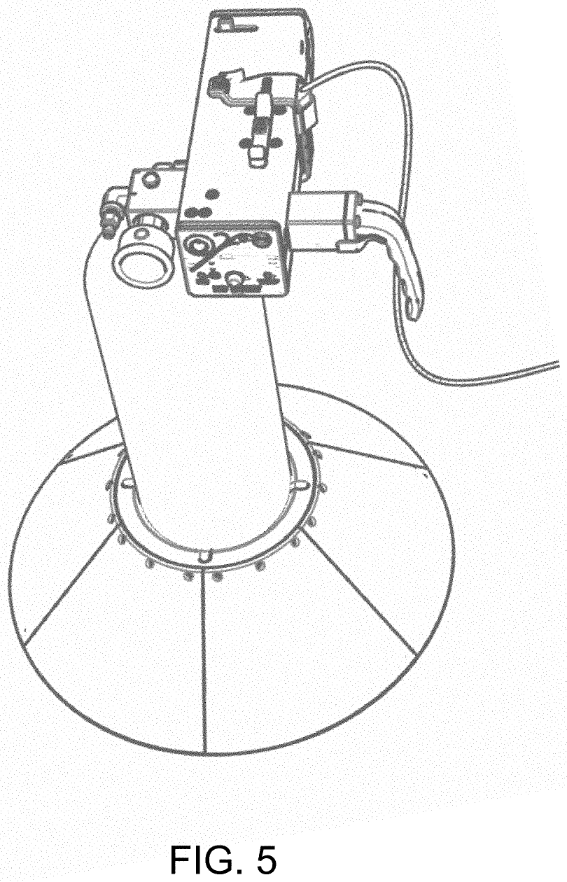

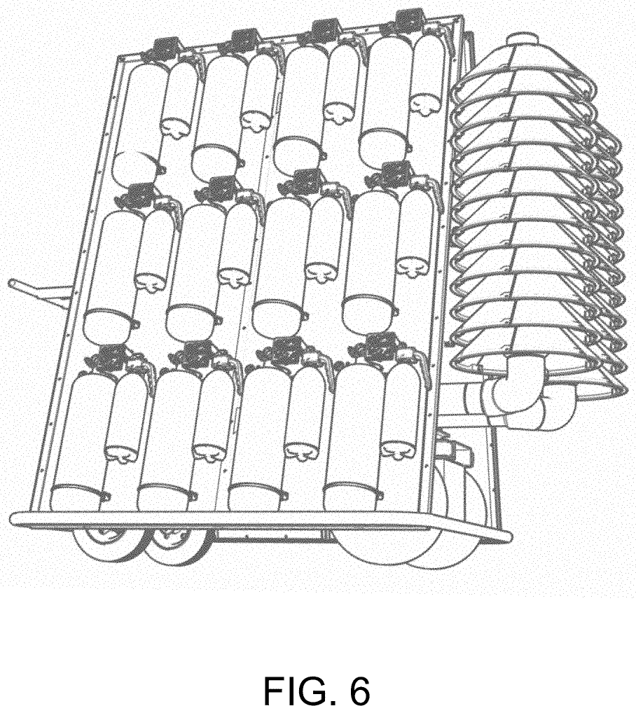

FIG. 5 shows the compressed air disinfection system (100) positioned on a stand (400). The stand may be made from different types of material. In some embodiments, the stand material may include a metal-based material, such as stainless steel, or a polymeric material, or a combination of metal-based and polymeric-based materials. The polymeric material may include acrylic-based polymers, such as acrylate, vinyl-based polymer, polyethylene-based polymers, such as PET or high density polyethylene (HDPE). The stand may be made by thermoforming processes, such as vacuum-forming. The stand may be placed inside of a vehicle and triggered either manually or remotely. As shown in FIG. 6, multiple stands (400) can be nested for storage fitted on the system distribution cart for transportation. A permanently mounted clip inside of the vehicle may be inserted in the system to arm the data logger and sensors; and provide the RFID reader with tag information.

The following examples further illustrate the invention but, of course, should not be construed as in any way limiting its scope.

### EXAMPLE 1

This example demonstrates the application of a compressed air disinfection system installed in a movable space, such as a motor vehicle, trailer, train car, bus or helicopter or airplane, Alternatively, the system may be installed in rental and shared car fleet, public transport buses, school buses, ambulance, trucks, automobiles, autonomous vehicles, helicopters and airplanes. Yet other spaces where the system may be installed are spaces without access to power or interior spaces where noise emission is of importance, such as in hospitals and nursing homes.

Transportation systems are critical environments for routine disinfection, and can only be disinfected during vehicle's or plane's downtime. Traditionally, commuter buses and rail cars are cleaned while the vehicle is sitting in a yard, powered down. An operator then uses the motorized cart to provide a number of pre-filled ready to use compressed air disinfection systems, and attaches one to each rail car/bus outfitted with a receptacle, such as for example a hole in the wall accepting the locking mechanism and a hinged door to seal off the port when no system is inserted in the port and which may be magnetically closed, and once locked in its position would press start, initiating the disinfecting cycle, or would initiate the start remotely. Alternatively, if the compressed air disinfection systems is semi-permanently installed in the rail car or bus, an operator would attach a container with an amount of disinfectant sized to handle the volume of the interior of the railcar or bus, attach a cylinder of compressed air containing a sufficient volume of air to dispense the entirety of the disinfectant through a permanently installed nozzle inside the rail car or bus, and initiate the disinfection cycle. In that configuration, the support member housing nozzles, supply and connecting line, regulators, and start button is installed in the compartment with a hinged lid on the exterior of the vehicle with only two sockets receiving the cartridges exposed. When the disinfection cycle is completed, both the container of disinfectant and the cylinder of compressed air will be empty and available, once detached from the compressed air disinfection systems on the outside of the railcar or bus, to be refilled and reused. Alternatively, the system may be placed inside of a vehicle, triggering the start sequence either manually or remotely. This operation may require a specifically designed stand and trigger clip.

To further illustrate the system, a city bus may have an interior volume of about from 30 m³ to 100 m³, depending on the type of bus used, an assumed average interior volume being about 60 m³. Depending on the type of disinfectant agent used, a certain target concentration of that particular disinfectant agent in the air is desired to achieve sufficient decontamination and disinfection per treatment cycle. In this particular example, HALOMIST®, distributed by Halosil Int'l LLC, was used. The target concentration is 11.8 ml/m³ in accordance with the EPA registration for this product. Thus, in this sample, the amount of disinfecting agent is calculated by multiplying the space to be treated by the target concentration (60 m³×11.8 ml/m³=708 ml), and the air bottle size and operating pressures are chosen to ensure that all liquid is dispensed plus an additional 10-20% buffer to ensure the system performs under all environment conditions.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

What is claimed is:

1. A compressed air disinfection system for deactivating pathogens on all surfaces or in air within a space comprising:
a source of a liquid disinfectant or deactivating agent,
a compressed air reservoir,
and
a support member comprising
an atomizing nozzle, connected via a low pressure air supply line providing low pressure air and a disinfectant supply line,
wherein the low pressure air supply line is further connected to an air valve,
a first air pressure regulator, connected to the compressed air reservoir via a high pressure air supply line providing high pressure air,
optionally, the first air pressure regulator is further connected to an intermediate air pressure supply line providing intermediate pressure air connected to a second pressure regulator,
and a means to initiate a disinfection or decontamination cycle by operation of the air valve.

2. The system of claim 1, wherein the source of the liquid disinfectant or deactivating agent is a bottle, a canister, a container, a cylinder, or a tank, the bottle, canister, container, cylinder, or a tank is so dimensioned that an amount of disinfectant contained therein is sufficient for a exactly a single disinfection or decontamination treatment of the space.

3. The system of claim 2, wherein the compressed air reservoir is so dimensioned that the amount of disinfectant is consumed before an amount contained in the compressed air reservoir is consumed.

4. The system of claim 1 further comprising a control system detecting operating conditions inside the space; and a data logging device powered by a rechargeable battery.

5. The system of claim 1, wherein the atomizing-nozzle is selected form the group consisting of a siphon nozzle, a gravity-fed nozzle, an internal or external mix nozzle, and a hydraulic nozzle.

6. The system of claim 5, wherein the atomizing-nozzle is a siphon nozzle.

7. The system of claim 1, wherein the support member is configured for a through-wall installation.

8. The system of claim 7, wherein the support member further comprises a locking mechanism a safety latch, an omnidirectional warning light, a keyed locking mechanism activator, a low temperature warning light, a pressure gauge indicating the nozzle air pressure, and a high humidity indicator.

9. The system of claim 8, further comprising a stand.

10. The system of claim 1, wherein the system does not contain an electrical power supply.

11. A rail car, vehicle or plane comprising the system of claim 1.

12. A method for deactivating contaminants within a space comprising installing one or more compressed air disinfection systems of claim 1, and initiating a decontamination or disinfectant treatment cycle.

13. The method of claim 12, wherein the high pressure air has a pressure of at least from 2000 psi to 10000 psi, the intermediate pressure air has a pressure of at least from 100 to 500 and the low pressure air had a pressure of at least from 20 psi to 45 psi.

14. The method of claim 12, wherein the volume of the enclosures to be treated are within 5 m3 to 300 m3.

15. A method of deploying multiple systems of claim 1 and treating a space from 300 m3 to 3000 m3.

16. The method of claim 15, wherein a disinfectant concentration within the space is from 5 ml/m³ to 15 ml/m³.

17. The method of claim 12, wherein in the liquid disinfectant or deactivating agent comprises HALOMIST®.
